(19) United States
(12) Patent Application Publication
(10) Pub. No.: US 2008/0249462 A1
(43) Pub. Date: Oct. 9, 2008

(54) Disposable Dispensers for Personal Use

(75) Inventor: Nilufer Smith, London (GB)

Correspondence Address: DENNISON, SCHULTZ & MACDONALD, 1727 KING STREET, SUITE 105, ALEXANDRIA, VA, 22314, US

(73) Assignee: NILUFER SMITH INNOVATIONS LIMITED, London (GB)

(21) Appl. No.: 12/090,881

(22) PCT Filed: Oct. 20, 2006
(86) PCT No.: PCT/GB06/03920

§ 371 Date: Apr. 21, 2008

(30) Foreign Application Priority Data
Oct. 21, 2005 (GB) 0521501.7
Mar. 31, 2006 (GB) 0606544.5

(51) Int. Cl. A61M 5/50; A61M 5/30

(52) U.S. Cl. 604/60; 604/110

(57) ABSTRACT

A syringe in accordance with the invention comprises a cylinder (10) having a smooth, substantially hemi-spherical ‘closed’ end (12) with a centrally placed hole (14) for dispensing the contents of the syringe. The plunger may have an annular rim (22) close to its inner end, which is arranged to engage with a corresponding annular slot (28) formed in the inner wall of the cylinder when the syringe is in its filled state. The rim (22) not only holds the plunger 16 in position prior to use, but also acts to prevent leakage of the contents. As the plunger (16) is depressed the rim (22) also acts to provide a tight seal between the piston and the cylinder. The cylinder has a second internal slot or groove (30) close to its domed end (12). When the plunger reaches the end of its travel the rim (22) engages tightly with the second annular slot (30) in order to prevent the piston being pulled out again for re-use of the syringe. The syringe is arranged so that it can be supplied filled with a suitable lubricant with its plunger ready for use without further manipulation.

This invention relates to disposable dispensers for personal use, and particularly to those for disposing a gel, viscous liquid or cream, lubricant or solids suspension. For the purposes of brevity, such gel, viscous liquid or cream, lubricant or solids suspension will be referred to herein as “lubricant” or “lubricants” as the context requires.

At one time in their lives nearly 75% of all women suffer at one time in their lives from vaginal dryness. Many products exist to treat this condition, but no convenient and suitable dispenser has been found to dispense such a product hygienically and comfortably.

Equally, a similar proportion of the population suffers at one time of their lives from haemorrhoids. Although the ultimate treatment for this condition is by surgery, many people have to wait for a considerable time before they can be treated. During this time a common way of attenuating the discomfort is by applying a gel or lubricant externally. Again no ideal dispenser has been suggested.

In accordance with the invention a single use syringe which is intended to be pre-filled and supplied to a user in a ready-to-use state without additional manipulation for applying a measured dose of a lubricant or the like to an intimate part of the body without the use of a needle, comprises a cylinder of a plastics material and arranged so that it can contain a personal lubricant, and a closely fitting plunger characterised in that the ‘closed’ end of the cylinder is smooth and domed for example, substantially hemi-spherical, ovaloid or ellipsoid, and devoid of any outer protuberances, or ridges or edges and is provided with a hole for dispensing the lubricant and the plunger is formed in a single piece which when fully depressed in the cylinder snugly fits the latter so that substantially all the contents of the syringe are expelled. The domed end of the body may be provided with a closely fitting removable cap to prevent leakage of the fluid from the syringe prior to use. To preserve the integrity of the package prior to use it may be provided with a tamper-proof seal.

By designing the syringe with a smooth, domed end for dispensing the lubricant it simplifies use of the device and avoids causing discomfort to the user whilst allowing the contents to be dispensed hygienically. The cylinder is arranged to contain a single dose, which may be from 1 ml to 5 ml, but in general it has been found that a dose of 1 to 2 ml I adequate. 1.6 ml is the preferred dose.

The invention relates to the particular features of the syringe. However, prior to use it is intended to be pre-filled with a suitable lubricant, such as a water soluble propylene glycol. Thus the syringe will be manufactured and may be filled either by the manufacturer or sold for filling elsewhere prior to sale to end-users in a state in which it is ready to use directly without further manipulation other than removing a tamper-proof seal and/or end cap, if present.

Whatever the circumstances, the treatment of either of the conditions mentioned above is highly personal and needs to be carried out as discreetly and comfortably as possible. The use of a syringe in accordance with the invention vastly simplifies the procedure used traditionally whereby a small reservoir is connected to an elongated tube with quite a sharp end. In use the end of the tube is cut or broken off and the contents ejected from the reservoir by squeezing it firmly. If the tube is sufficiently flexible not to be painful, it tends to bend and may obstruct the flow of the lubricant. It may also have a sharp edge where the tube is cut open or broken.

A syringe in accordance with the invention has none of these disadvantages, is easy to insert and simple to operate without causing either pain or discomfort. More particularly, it allows its contents to be dispensed exactly where they are needed in a single measured shot, after the syringe can be disposed of hygienically. Moreover, the contents of the syringe can be dispensed hygienically without the user's fingers coming into contact either with the product or the body.

The plunger advantageously has a locating element close to its inner end, which is arranged to engage with a corresponding slot or groove on the inner surface of the cylinder to hold the plunger in the filled state of the syringe until use. They cylinder also has a second slot or groove close to its inner end and arranged to engage the locating element on the plunger in its depressed state once the device has been used in order to prevent re-use of the syringe.

The locating element at the inner end of the plunger preferably comprises an annular rim or ridge which sealingly engages with a corresponding annular slot the inner wall of the cylinder in its filled state, which not only holds it in position, but also acts to prevent any leakage of the contents. It has a second slot or groove close to its inner end which engages firmly with the locating element on the plunger when the latter is depressed in order to prevent re-use of the syringe.

The cylinder has a flange at its outer end to facilitate gripping the cylinder during use. The outer end of the plunger is formed with an enlarged button or annulus to make it easy to press. When the plunger is fully depressed the button advantageously fits snugly into a corresponding depression in the flange at the outer end of the cylinder in order to make it difficult or impossible to re-use the syringe.

The inner end of the plunger is advantageously of a substantially hemispherical form in order substantially to mate with the inner end of the cylinder so that in use the entire contents of the syringe can be ejected in a single stroke. The syringe is thus dimensioned accordingly.

The length of the syringe is such that when inserted appropriately contents is ejected at the required depth without the user's fingers becoming soiled. Typically, the length of the cylinder is between 5 and 10 cm, and preferably between 6 and 7 cm so that the overall length, including the plunger is between 7 and 15 cm.

Precision manufacturing allows the plunger to be moulded as a single piece with the annular rim in view of the fact that the intended contents of the syringe will generally be viscous or even paste-like at ambient temperature. A suitable plastics material for the manufacture of the syringe parts is a polystyrene, preferably of an impact grade or polyethylene. The syringe may either be transparent, though in most cases it is preferred that it is opaque as occasionally air bubbles may be present in the syringe once filled. The plunger may be made of a coloured plastics material for decoration or coding of the contents.

By manufacturing the cylinder, the plunger and the protective cap, if present, each as a single moulding makes the syringe simple to assemble during manufacture and filling, and results in a cheap and highly effective device.

The contents of the syringe are dispensed from the hole at the hemispherical end of the cylinder. The hole is between 1 and 3 mm, and preferably 2 mm in diameter as dictated by the viscosity of the intended contents of the syringe.

A syringe in accordance with the invention may also find applications in respect of either heterosexual or homosexual practices. In such circumstances, the lubricant or contents may or may not be medicated, for example, to contain spermicides or an anti-AIDS medication or the like.

A syringe in accordance with the invention will now be specifically described with reference to the accompanying drawings, in which:

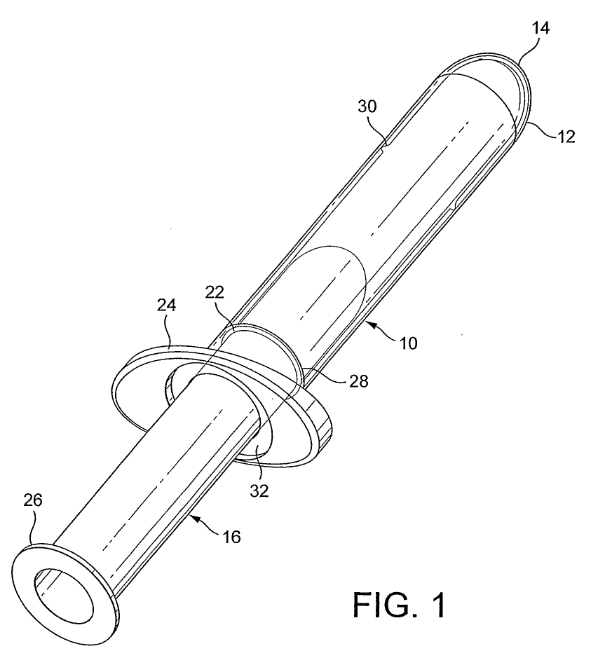

FIG. 1 is an isometric view of the syringe assembled as it would be supplied,

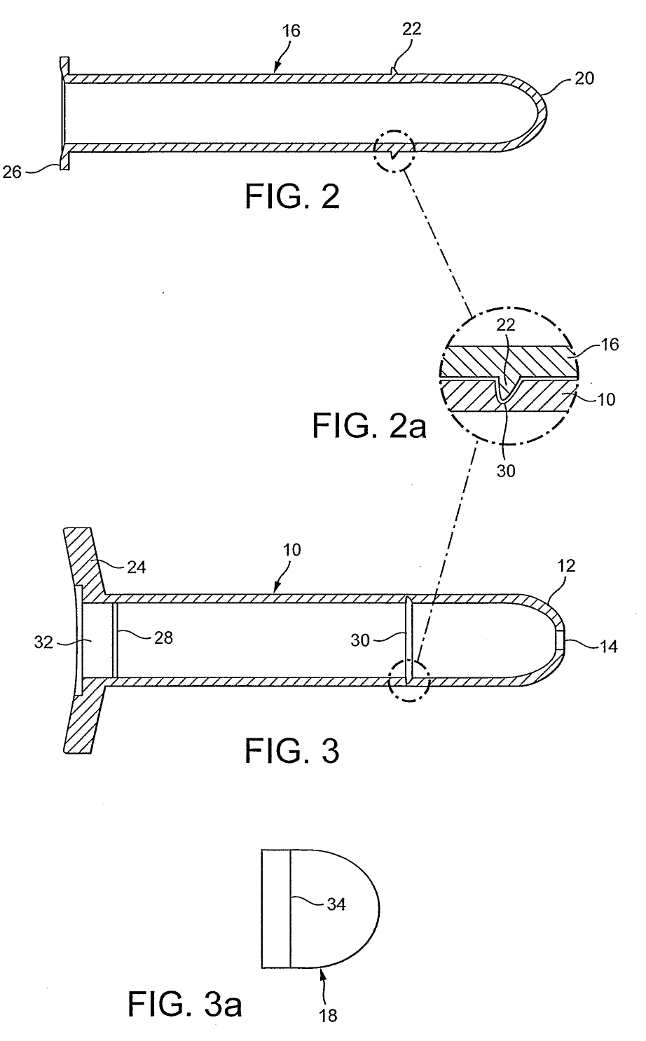

FIG. 2 is a sectional side elevation of the plunger of the syringe,

FIG. 2a is a detailed section of the wall of the syringe,

FIG. 3 is a sectional side elevation of the plunger of the syringe,

FIG. 3a shows a closure cap for the syringe, and

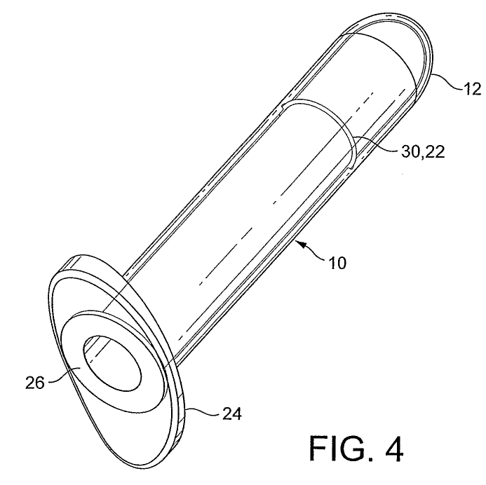

FIG. 4 is an isometric view of the syringe once the plunger has been depressed to dispense the contents of the cylinder.

The syringe comprises a cylinder 10 whose ‘closed’ end 12 is smooth and domed, for example, substantially hemi-spherical, ovaloid or ellipsoid, and devoid of any outer protuberances, or ridges or edges. It is has a centrally placed hole 14 for dispensing the contents of the syringe. Once filled with the contents, as shown in FIG. 1, where it is ready for use, the cylinder is closed by a moveable plunger 16.

Normally the hole 14 at the end of the syringe would be closed by a heat sealed or adhesive tab (not shown) which can be peeled off by the user. An optional closure cap 18 is shown separately (FIG. 3a). When the syringe is supplied to a user, already factory-filled with a suitable lubricant and ready for use the cap 18 would be in position over the outer end 12 of the syringe to prevent leakage of the lubricant. The cap 18 is further provided with an inner annular rim 34 that holds it in place over the end of the cylinder 12 and acts as a further barrier to prevent possible leakage of the contents of the syringe. A tamper-proof seal (not shown) would normally be provided.

By designing the syringe with a smooth, domed end for dispensing the lubricant it simplifies use of the device and avoids causing discomfort to the user whilst allowing the contents to be dispensed hygienically. A suitable choice of plastics material, such as some blends of polyethylene, have a soft touch which is preferred.

The plunger 16, as shown in FIG. 2 separately from the cylinder in FIG. 3, is formed as a single moulding and fits closely within the bore of the cylinder 10 and has a substantially hemi-spherical shaped inner end 20.

The plunger has an annular rim 22 close to its inner end, which is arranged to engage with a corresponding annular slot or groove 28 formed in the inner wall of the cylinder when the syringe is in its filled state. The engagement of the rim 22 in the outer slot 28 not only holds the plunger 16 in position, but also acts to prevent leakage of the contents. As the plunger 16 is depressed the rim 22 also acts to provide a tight seal between the plunger and the cylinder and to prevent any flow-back of the contents.

The cylinder has a second internal annular slot or groove 30 close to its inner, domed end 12. When the plunger reaches the end of its travel the rim 22 engages tightly with the second annular groove 30 in order to prevent the piston being pulled out again for re-use of the syringe. This is shown more clearly in FIG. 4 where the plunger 16 is fully depressed expelling substantially all of the contents.

A detail ed drawing showing the mating of the rim 22 on the plunger with the annular groove 30 on the inner wall of the cylinder is shown in FIG. 2a. To ease the disengagement of the rim 18 on the plunger from the annular slot 28 in the cylinder, the forward face of the rim may be at a suitable sloping angle in the order of 30 to 60°, but preferably at 45°, whereas the rear face may be much steeper, nearing 90° or nearly perpendicular to the axis of the plunger. The faces of the corresponding grooves 28 and 30 in the inner surface of the cylinder may be at corresponding angles.

As a user may try to force the piston back once it has been used (and the rim 22 has engaged with the groove 30 in the cylinder) by inserting a rod or the like through the hole 14 in the cylinder, the inner end of the piston opposite the hole 14 may be weakened by making it thinner than the rest of the piston. It is thus strong enough only to withstand the pressure of the lubricant, but ruptures if any other pressure is exerted on it as an additional precaution against re-use of the syringe.

If required, and additional rim or latch (not shown) can be provided on the piston to engage with the outer groove in the cylinder as the inner rim 22 engages with the inner groove 30

The cylinder 10 has a flange 24 at its outer end to facilitate gripping the cylinder during use. The outer end of the plunger 16 is formed with an enlarged button or annulus 26 to facilitate use of the syringe. When the plunger is fully depressed the button 26 fits snugly into a corresponding depression 32 in the flange so that the outer surface of the button 26 is substantially flush with the outer surface of the flange 24 (FIG. 4). Coupled with the action of the rim 22 and the groove 30 the ‘burying’ of the button 26 on the top of the plunger makes it very hard to pull the plunger out again so that it is effectively impossible to re-use the syringe. The plunger 16 may also be provided with a ratchet or a series of barbs (not shown) which are arranged to lock progressively with the slot to prevent the plunger from being withdrawn even after only partial use.

The inner, domed end 20 of the plunger 16 is of a substantially hemispherical form in order substantially to match that of the domed inner end of the cylinder 10 so that in use the entire contents of the syringe can be ejected in a single stroke.

The fact that the cylinder, the plunger and the protective cap, if present, are formed each as a single moulding facilitates manufacture makes the syringe simple to assemble during manufacture and filling, and results in a cheap and highly effective device.

The syringe is intended for a single use to apply a measured dose of a lubricant or the like to an intimate part of the body without the use of a needle. Whatever the circumstances, the treatment of either of vaginal dryness or haemorrhoids or for other personal use needs to be carried out as discreetly and comfortably as possible. The use of a syringe device in accordance with the invention which is ready for immediate use without further manipulation vastly simplifies the procedure over that which is possible with a traditional tube device in which the contents are ejected by squeezing it firmly. Furthermore, as the totality of the contents of the tube is expelled the risk that any remainder of the contents may cause soiling is greatly reduced.

The contents of the syringe are dispensed from the hole 14 at the end of the cylinder. The hole is between 1 and 3 mm, and preferably 2 mm in diameter depending on the viscosity of the intended contents of the syringe. In some applications it may be advantageous to place an axial slot or slots in the end of the cylinder instead of a single hole at the centre of the dome so that the contents of the cylinder are dispersed more evenly. This also makes it more difficult to push the plunger back to permit re-use of the syringe.

The syringe will generally need to have an internal volume of between 1 and 5 ml, and ideally of 1½ to 2 ml. The cylinder is thus dimensioned accordingly having an external diameter of between say 6 mm and 12 mm, and probably about 9 mm.

The length of the syringe is also such that when inserted appropriately its contents are ejected at the required depth without the user's fingers becoming soiled. Typically, the length of the cylinder is between 5 and 10 cm, and preferably between 6 and 7 cm so that the overall length, including the plunger is between 7 and 15 cm.

A syringe in accordance with the invention is easy to insert and simple to operate without causing either pain or discomfort, and is cheap enough to be discarded after use without hesitation. Because the plunger fits snugly into the cylinder once the contents have been expelled, there is almost no risk of a leak of any liquid.

Such a syringe is discreet enough to be kept safely in a handbag or a pocket, and is suitable for being sold in packs of six, ten or twelve, or as required.

1. A single use syringe for applying a measured dose of a lubricant (as herein defined) to an intimate part of the body without the use of a needle, comprising a cylinder of a plastics material arranged to contain the lubricant and a closely fitting plunger characterised in that the outer surface of the ‘closed’ end of the cylinder is smooth and gently domed, devoid of any outer protuberances, or ridges or edges and is provided with a hole for dispensing the lubricant, and the plunger is formed as a single moulding and is shaped so that when fully depressed it forms a snug fit with the inside of the cylinder and has means for locking it in place to prevent re-use of the syringe.

2. A syringe as claimed in claim 1 in which the plunger has an annular rim or ridge close to its inner end which is arranged to engage with a corresponding slot or groove on the inner surface of the cylinder to hold the plunger in its filled state until use, and a second slot or groove in the cylinder close to its inner end and arranged to engage the annular rim on the plunger in its depressed state to prevent re-use of the syringe.

3. A syringe as claimed in claim 1 in which the piston or the cylinder is provided with a ratchet series of barbs which engage respectively on the other member to prevent the plunger from being drawn outwardly even when it has been only partially depressed.

4. A syringe as claimed in claim 1 in which, the forward face of the rim on the plunger may be at a sloping angle in the order of 30 to 60°, preferably at 45°, and the rear face is nearly perpendicular to the axis of the plunger and the faces of the corresponding grooves 28 and 30 in the inner surface of the cylinder are at corresponding angles.

5. A syringe as claimed in claim 4 in which the cylinder has a flange at its outer end to facilitate gripping the cylinder during use, and
the outer end of the plunger is formed with an enlarged button or annulus which fits a corresponding depression in the outer end of the flange when the plunger is fully depressed in order to make it difficult or impossible to re-use the syringe.

6. (canceled)

7. A syringe as claimed in claim 1 in which the inner end of the plunger is of a substantially hemispherical shape in order to mate with the inner end of the cylinder so that in use the entire contents can be ejected from the syringe.

8. A syringe as claimed in claim 1 in which the inner end of the plunger has a weakened area opposite the discharge hole in the cylinder, which is designed to rupture if an attempt is made to push the plunger back out of the cylinder.

9. A syringe as claimed in claim 1 in which the end of the cylinder is provided with an axial slot or slots in instead of a single hole at the centre of the dome so that the contents of the cylinder can be dispersed more evenly.

10. A syringe as claimed in claim 1 in which the hole in the end of the cylinder is between 1 and 3 mm.

11. A syringe as claimed in claim 1 in which the cylinder has an external diameter of between 6 mm and 12 mm.

12. A syringe as claimed in claim 1 in which the syringe has an internal volume of between 1 and 5 ml.

13. (canceled)

14. A syringe as claimed in claim 12 in which the syringe has an internal volume of 1½ to 2 ml.
